(19) United States
(12) Patent Application Publication
(10) Pub. No.: US 2005/0101958 A1
(43) Pub. Date: May 12, 2005

(54) Bone fixing system

(76) Inventor: Michael Adam, Merlischachen (CH)

Correspondence Address: CHRISTIE, PARKER & HALE, LLP, PO BOX 7068, PASADENA, CA, 91109-7068, US

(21) Appl. No.: 10/667,248

(22) Filed: Sep. 17, 2003

(30) Foreign Application Priority Data
Nov. 4, 2002 (EP) 02 024 606.2

(51) Int. Cl. A61B 17/72

(52) U.S. Cl. 606/64; 606/62

(57) ABSTRACT

The invention relates to a bone fixing system comprising at least one nail, in particular a femoral medullary nail, and at least one screw, in particular a condyle screw, which can be guided through a transverse bore formed in the nail and defining the orientation and the position of the screw with respect to the longitudinal axis of the nail, with at least one clamping member being provided which can be introduced into a longitudinal bore of the nail and is axially adjustable in the longitudinal bore relative to the nail, with the screw guided through the transverse bore of the nail being able to be clamped between the clamping member and the inner wall of the nail bounding the transverse bore by the displacement of the clamping member.

The invention relates to a bone fixing system comprising at least one nail and at least one screw which can be guided through a transverse bore formed in the nail and defining the orientation and the position of the screw with respect to the longitudinal axis of the nail.

Such systems in particular serve for the repositioning of distal femoral fractures such as are shown, for example, on page 141 in the “Manual der Osteosynthese” (Manual of Osteosynthesis), 3rd edition, Springer-Verlag, Authors: M. E. Müller, M. Allgöwer, R. Schneider, H. Willenegger. The nail is in particular a femoral medullary nail which is inserted from the side of the knee joint and which can be placed onto a target apparatus which allows the transverse bores formed in the nail to be located by a drill at the angle pre-determined by the target apparatus and corresponding to the orientation of the transverse bores and pre-bores to be made in the bone for the screws to be subsequently inserted. These screws, which are in particular provided in the form of condyle screws, serve to fix the medullary nail in the bone with respect to its axial direction.

Such a fixing system is also used when condyle fragments should be fixed with respect to the remaining bone by means of the screws inserted through the transverse bores of the nail. The medullary nail, which is held in the remaining bone and is fixed by means of locking screws there, in particular represents the only fixed reference basis for the fixing of the condyle fragments when the respective fracture is characterized by a plurality of condyle fragments. Examples for such fractures are shown in FIGS. C1, C2 and C3 on page 141 in the aforesaid “Manual der Osteosynthese”.

It has been found to be problematic with such fractures that the bone screws serving for the fixing of the condyle fragments and guided through the transverse bores of the medullary nail are substantially freely movable with respect to their own axial direction relative to the medullary nail.

A locking nail is known from DE 200 12 877 U1 which has a transverse bore for a bone screw, said transverse bore having an elongate shape in the longitudinal direction. The bone screw can be displaced in the transverse bore in the axial direction of the locking nail, and indeed by means of a locking member which has an outer thread section and can be screwed in an inner thread of the locking nail which is hollow in the region of the elongate transverse bore.

The goal is likewise pursued in DE 296 20 327 U1 of being able to change the position and the orientation of locking screws with respect to a locking nail. For this purpose, the locking nail is provided with an elongate opening extending in the longitudinal direction of the nail shank. Furthermore, so-called sliding blocks can be introduced into the nail shank, with the intermediate spaces between the sliding blocks serving as passages for the locking screws.

In contrast to this prior art, the invention starts from a bone fixing system comprising stable angle screws whose orientation and position are each defined by the transverse bore of the nail.

It is the object of the invention to further develop a bone fixing system of the kind initially mentioned such that the nail can be fixed reliably and permanently in the most simple and secure manner possible, with it in particular being possible, in the repositioning of distal femoral fractures, to reliably fix a plurality of condyle fragments in their correct position with respect to the remaining bone.

This object is satisfied by the features of claim 1 and in particular in that at least one clamping member is provided which can be introduced into a longitudinal bore of the nail and is axially adjustable in the longitudinal bore relative to the nail, with the screw guided through the transverse bore of the nail being able to be clamped between the clamping member and the inner wall of the nail bounding the transverse bore by the displacement of the clamping member.

In accordance with the invention, the screw inserted through the transverse bore of the nail can be fixed in the respectively desired penetration depth relative to the nail simply by adjustment of a clamping member introduced into the longitudinal bore of the nail and can be fixed in this manner with respect to its axial direction, and indeed while maintaining its orientation and position with respect to the longitudinal axis of the nail defined by the transverse bore of the nail. It is here of particular advantage that the penetration depth of the screw is freely selectable, i.e. the screw can be fixed in any penetration depth by means of the clamping member.

It is furthermore advantageous for the clamping member in accordance with the invention to be usable with conventional screws such that no specifically designed screws are required. Furthermore, it is of advantage in accordance with the invention that a longitudinal bore present in the nail, by means of which the nail can be guided on a so-called Kirschner wire, is simultaneously usable for the clamping member in accordance with the invention.

In a preferred embodiment of the invention, the longitudinal bore of the nail is provided with an inner thread section in which the clamping member can be screwed. The fixing of the screw can here be simply brought about by a screw movement of the clamping member.

In a preferred practical embodiment, the clamping member is made in one part and is in particular provided in the form of a grub screw.

In a further preferred embodiment of the invention, at least one sleeve-like or bushing-like insert is introduced into the longitudinal bore of the nail and has at least one passage aligned with the transverse bore of the nail and with which the clamping member cooperates. The inner side of the insert is preferably provided with an inner thread section in which the clamping member can be screwed.

A direct cooperation is hereby avoided between the clamping member, on the one hand, and the nail, on the other hand. This is in particular of advantage when the nail is made of a material for which the manufacture and the use of a thread for the clamping member is problematic, in particular due to too low a material hardness. Titanium or a titanium alloy is preferably usable for the material of the nail due to the good biocompatibility. The design and use of a thread are problematic with such comparatively soft materials, above all with relatively long and simultaneously fine threads such as can be used nails in accordance with the invention.

For this reason, in accordance with a further embodiment, the insert is made of a material which has a higher toughness and/or hardness than the material of the nail. A cobalt chromium alloy can, for example, be used for the material of the insert.

The insert is preferably connected rotationally fixedly to the nail. The insert is preferably pressed or screwed into the longitudinal bore of the nail.

In accordance with a further preferred embodiment of the invention, a plurality of transverse bores are made in the nail, with a clamping member being provided for each screw which can be guided through one of the transverse bores. An individually adjustable clamping can hereby be realized for the individual screws.

Provision is furthermore preferably made for a set of different axial spacings to be provided between the nails having transverse bores and for the axial length of the clamping members to be smaller than the smallest axial spacing between two sequential transverse bores occurring in the set.

It has been found that a secure and permanent fixing of the screws in the transverse bores of the nail can even be achieved with comparatively short clamping members. It is hereby possible to provide only one length type of clamping members for the whole set of nails and also to use nails with comparatively low axial spacings between sequential transverse bores.

In a further embodiment of the invention, provision is made for a section of the clamping member disposed on the side of the screw remote from the adjustment device to be movable toward the screw.

In this variant of the invention, an adjustment device for the clamping member is consequently provided with which the clamping member can be drawn toward the screw.

Here, the clamping member is preferably freely movable at least in the axial direction in the longitudinal bore of the nail and is provided with at least one passage for the screw which can be aligned with the transverse bore of the nail. The clamping member is preferably of sleeve shape.

A particularly simple actuation of the clamping member is achieved if, in accordance with a further embodiment of the invention, the adjustment device includes a drawing screw which cooperates with a threaded section of the clamping member and is supported at the nail for the drawing of the clamping member in the axial direction. The clamping member can here be drawn in the direction of the drawing screw by rotating the drawing screw and can be moved against the screw inserted through the transverse bore of the nail in order to clamp the screw.

Provision is preferably made for the clamping member to have a plurality of passages spaced from one another in the axial direction and each alignable with a transverse bore of the nail. Here, the screws each extend simultaneously through the transverse bore formed in the nail and through the passage provided in the clamping member such that a plurality of screws can be fixed by a single clamping member by an axial displacement of the clamping member, in particular by drawing at the clamping member by means of a drawing screw.

The fixing of all screws is ensured in a particularly simple and simultaneously reliable manner if, in accordance with a further preferred embodiment of the invention, the clamping member can be deformed in the axial direction by means of the adjustment device. It is hereby not necessary for all screws to come into engagement with the clamping member at exactly the same point in time in order to be clamped securely between the clamping member and the inner wall of the nail bounding the respective bore. This tolerance achieved by the axial deformability of the clamping member ensures that all screws are fixed in their transverse bore with a sufficiently large clamping force.

Furthermore, in accordance with the invention, at least one securing member is preferably provided which can be moved from the outside through the side wall of the nail into its longitudinal bore and is fixable in its starting position by the clamping member relative to the nail prior to the actuation of the adjustment device.

The securing member, which is in particular provided in the form of a securing screw which can be screwed into the side wall of the nail, can here be designed such that it is deformed or sheared off by the subsequent axial displacement of the clamping member such that the securing member ensures a correct alignment of the transverse bores of the nail with the passages of the clamping member for the guiding through of the screws, on the one hand, but does not interfere with the subsequent axial displacement of the clamping member for the fixing of the screws, on the other hand.

Further embodiments of the invention are recited in the dependent claims, in the description and in the drawing.

The invention will be described in the following by way of example with reference to the drawing. There are shown:

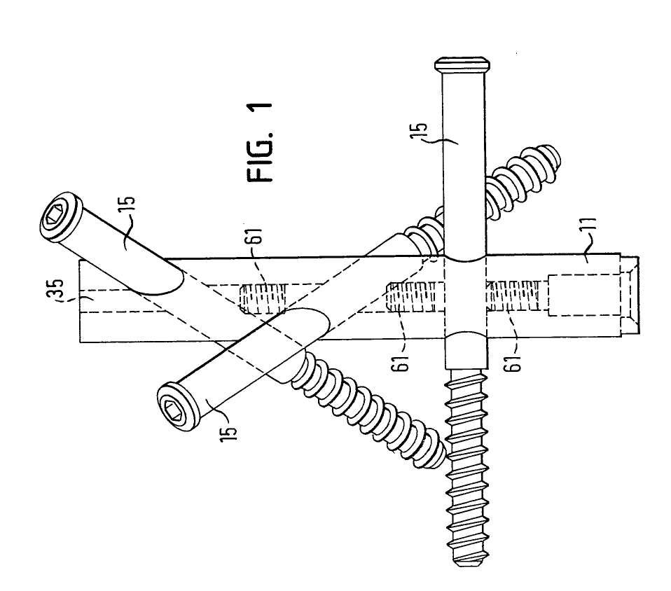

FIG. 1 a bone fixing system in accordance with a first embodiment of the invention;

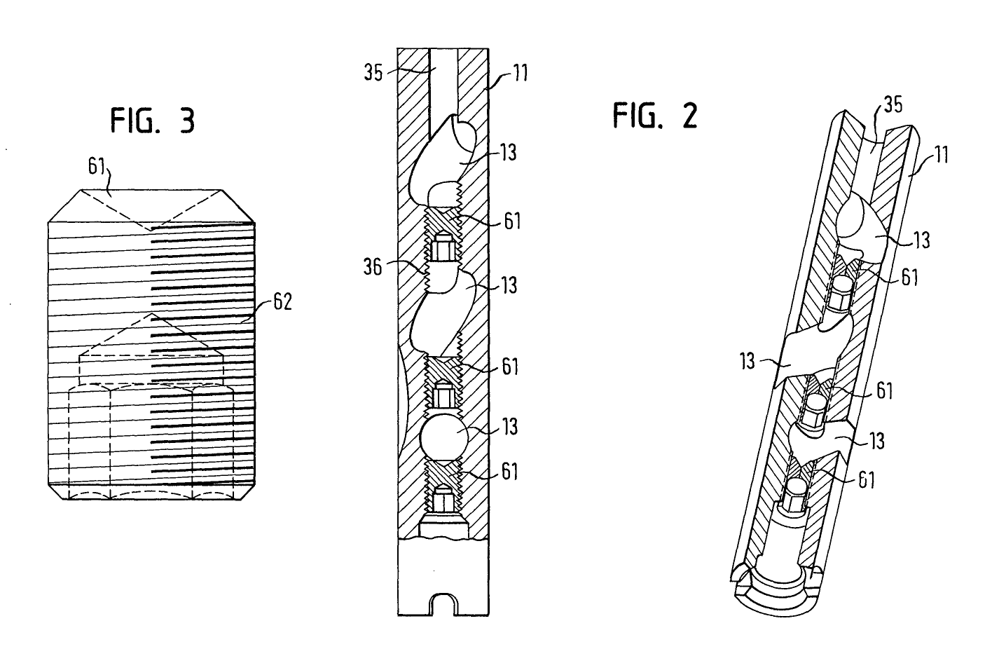

FIG. 2 various views of a nail of the system of FIG. 1 fitted with a plurality of clamping members;

FIG. 3 a clamping member of the system of FIG. 1;

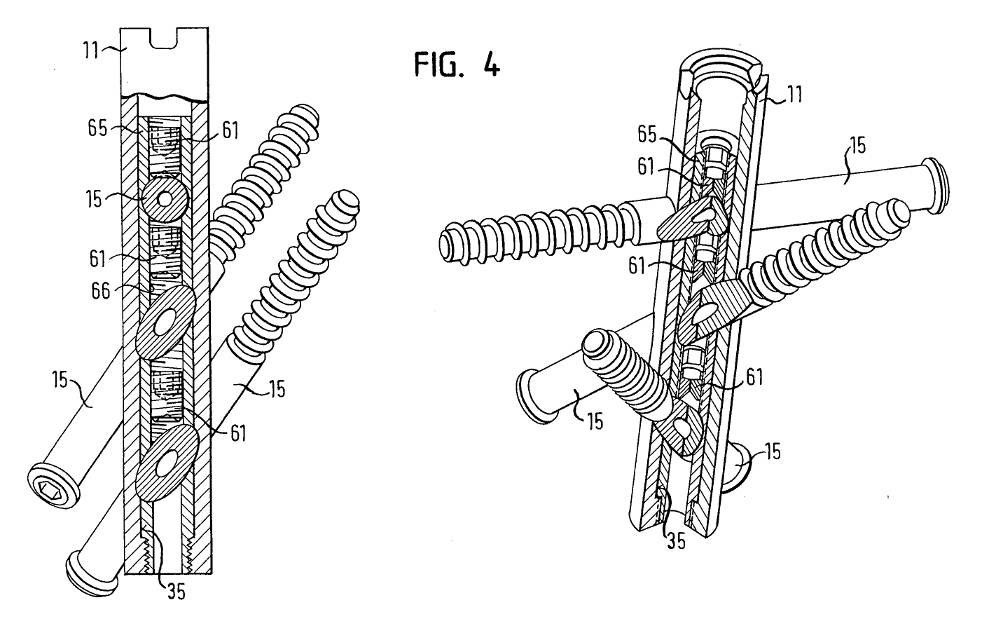

FIG. 4 various views of a bone fixing system in accordance with a second embodiment of the invention;

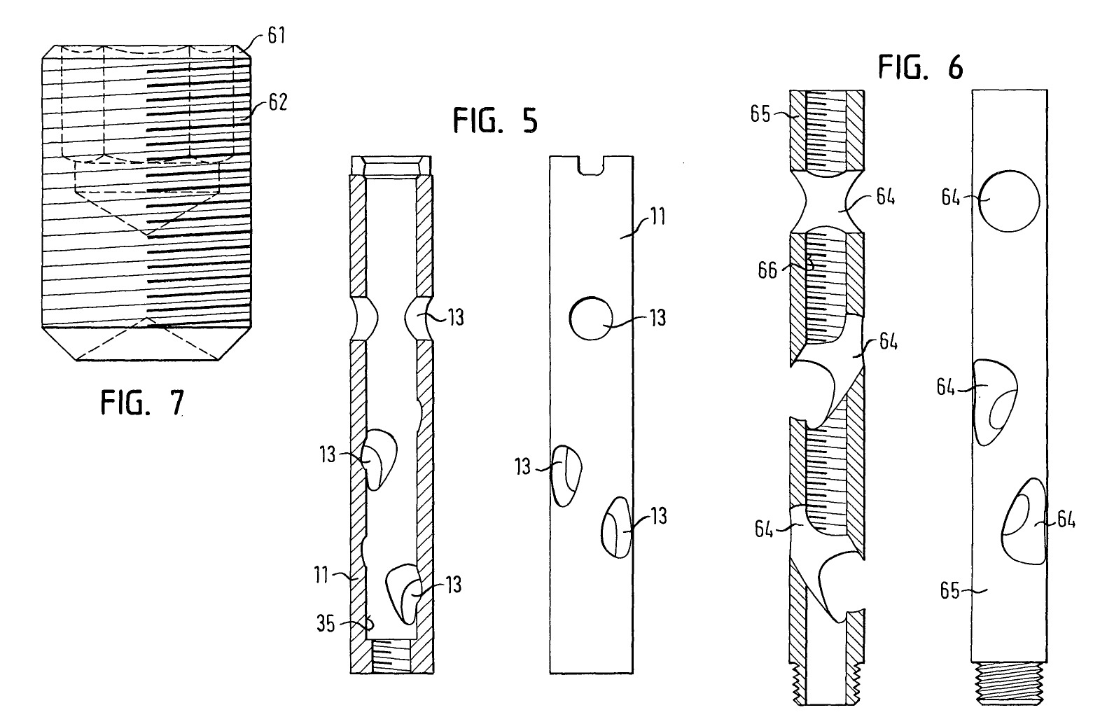

FIG. 5 various views of a nail of the system of FIG. 4;

FIG. 6 various views of a sleeve-like insert of the system of FIG. 4;

FIG. 7 a clamping member of the system of FIG. 4;

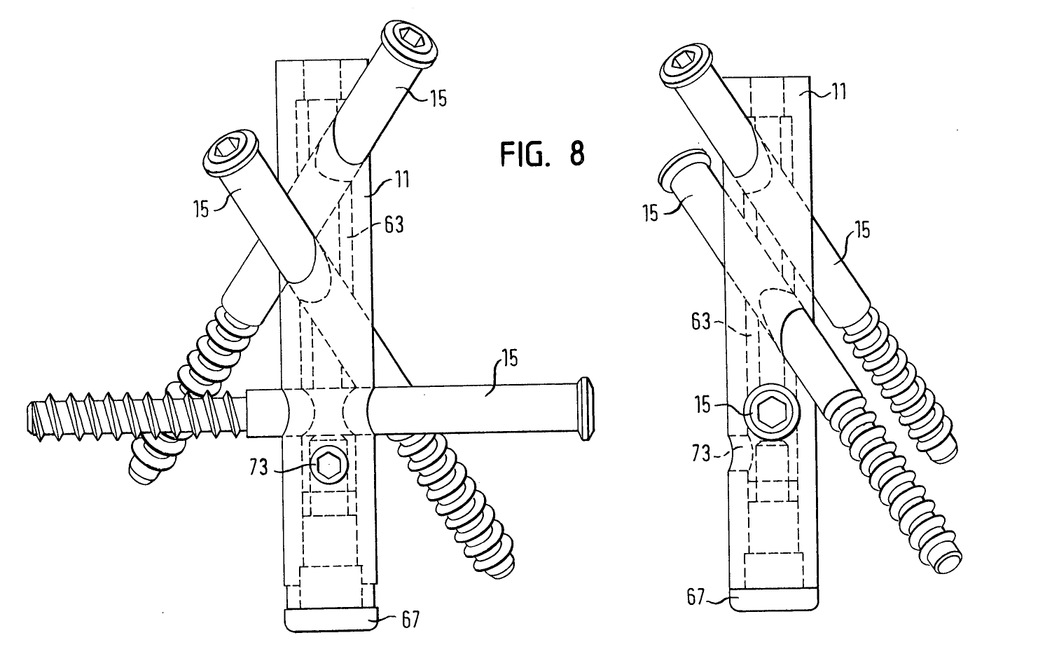

FIG. 8 various views of a bone fixing system in accordance with a third embodiment of the invention;

FIG. 9 various views of a nail of the system of FIG. 8;

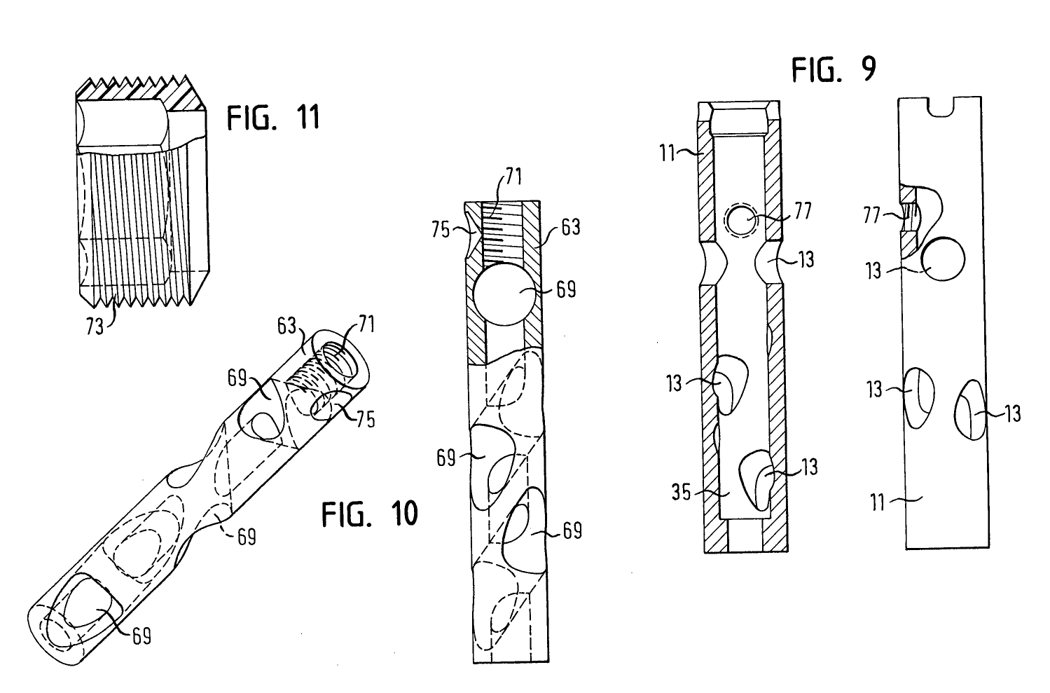

FIG. 10 various views of a sleeve-like clamping member of the system of FIG. 8;

FIG. 11 a securing member of the system of FIG. 8; and

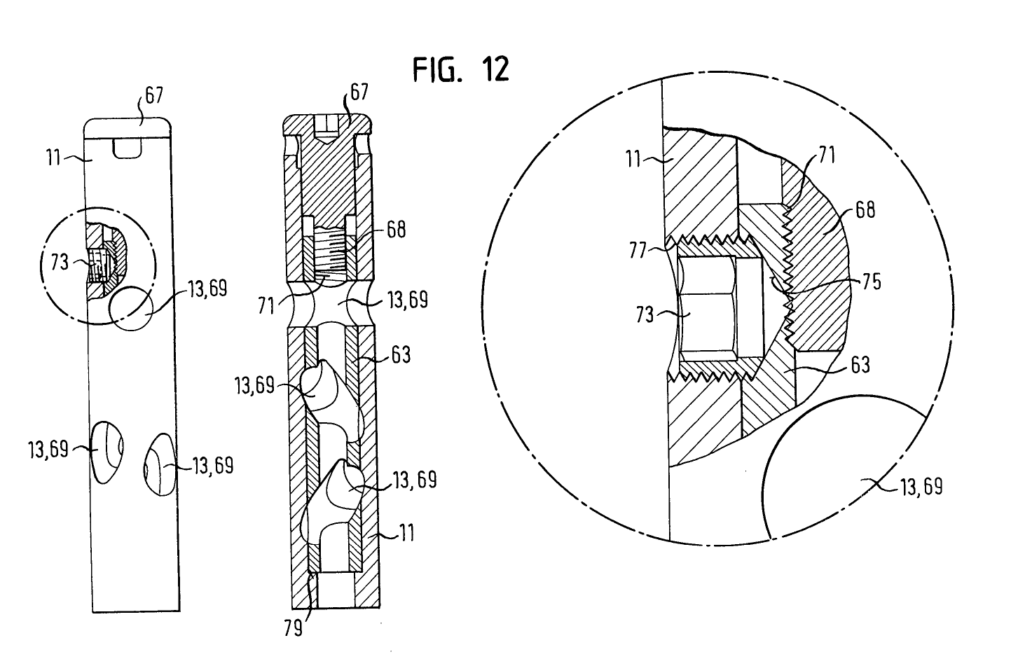

FIG. 12 various views of the system of FIG. 8 without condyle screws in the mounted state.

The bone fixing system in accordance with the invention shown in FIGS. 1 to 3 includes a femoral medullary nail 11 and a plurality of condyle screws 15 which are guided through transverse bores formed in the nail 11. The nail 11 has a central longitudinal bore 35 which is provided with an inner thread 36 via which individual clamping members 61 in the form of grub screws are screwed into the longitudinal bore 35 of the nail 11 to fix the respective condyle screws 15 in their desired axial position.

In FIG. 2, which shows the assembled state in section without the condyle screws, in particular the inner thread 36 of the nail 11 is shown with which the inner wall bounding the longitudinal bore 35 of the nail 11 is provided.

The grub screw 61, which is shown enlarged in FIG. 3 relative to FIG. 2 and which is provided with a corresponding outer thread 62, is actuable by means of a hexagon head tool.

The grub screws 61 are introduced via the lower end of the nail 11 in FIG. 2 into its longitudinal bore 35 and screwed in by means of the mentioned tool. In this variant of the invention, the order of the fixing of the condyle screws 15 is pre-determined since the condyle screw respectively disposed furthest away from the introduction end has to first be fixed by means of the relevant clamping member 61 before a further condyle screw 15 can be introduced into the transverse bore 13 of the nail next disposed.

If the nail 11 is not guided on a so-called Kirschner wire for the insertion of the nail 11 into the bone, the innermost grub screw 61, i.e. the uppermost in FIG. 2, can already be screwed into the nail 11 when this is inserted into the bone.

The second embodiment of the invention shown in FIGS. 4 to 7 differs from the variant explained above in that a sleeve-like insert 65 is introduced into the longitudinal bore 35 of the nail 11. The sleeve insert 65 can, for example, be pressed or screwed into the nail 11.

As is shown in FIG. 6, in which the sleeve insert 65 is shown enlarged with respect to the nail 11 shown in FIG. 5, the insert 65 is provided with passages 64 for the condyle screws 15, with the passages 64 of the insert 65 being aligned with the transverse bores 13 of the nail in the state in accordance with FIG. 4 introduced into the longitudinal bore 35 of the nail 11.

The inner wall of the sleeve insert 65 is provided with an inner thread section 66 into which the grub screws 61 (cf. FIG. 7) are screwed in order to fix the condyle screws 15 each extending through a passage of the insert 65 and a transverse bore 13 of the nail 11.

What was said in this respect in connection with the first embodiment in accordance with FIGS. 1 to 3 applies as regards the order of the fixing of the screws 15.

The sleeve insert 65 preferably consists of a material which is tougher or harder than the material of the nail 11. While the nail 11 is preferably made of titanium or a titanium alloy and is thus manufactured from a comparatively soft material, the sleeve insert 65 preferably consists of a cobalt chromium alloy.

Other material combinations can generally also be used in accordance with the invention.

A direct cooperation between the grub screws 61 and the nail 11 is avoided by the use of the sleeve insert 65, whereby a stable and reliable fixing of the condyle screws 15 is permanently ensured by the grub screws 61 irrespective of the material used for the nail 11.

Unlike in the previously explained variants, in the third embodiment of the invention shown in FIGS. 8 to 12, a single common clamping member 63 is provided for a plurality of condyle screws 15 in the form of a clamping sleeve 63 with which the condyle screws 15 can be fixed by a single axial displacement of the clamping sleeve 63 and thus practically simultaneously.

The clamping sleeve 63, which is shown enlarged in FIG. 10 with respect to the nail 11 shown in FIG. 9, is insertable into the longitudinal bore 35 of the nail 11 and axially freely movable therein. The clamping sleeve 63 is provided with passages 69 which can be aligned with the transverse bores 13 formed in the nail 11. The correct alignment both with respect to the axial position and with respect to the rotational position of the clamping sleeve 63 relative to the nail 11 is ensured by a securing member 73 in the form of a securing screw shown enlarged in FIG. 11.

The side wall of the nail 11 is provided with a passage 77 for the securing screw 73, said passage having an inner thread section into which the securing member 73 having a corresponding outer thread can be screwed such that the securing member 73 projects into the longitudinal bore 35 of the nail 11 and enters into engagement with a recess 75 of the clamping sleeve 63 provided for the securing member 73.

The securing serving for the correct alignment of the clamping sleeve 63 relative to the nail 11 is shown in particular in the enlarged representation at the far right in FIG. 12.

The axial displacement of the clamping sleeve 63 for the simultaneous fixing of the condyle screws 15 guided through the transverse bores 13 of the nail 11 and through the passages 69 of the clamping sleeve 63 (cf. FIG. 8) is brought about by an adjustment device 67 in the form of a drawing screw which is screwed in an inner thread section 71 of the clamping sleeve 63 (cf. FIG. 10) via a drawing section 68 provided with an outer thread and whose head is supported at the front face of the nail 11.

By rotating the drawing screw 67, the clamping sleeve 63 is drawn in the direction of the drawing screw 67. All condyle screws 15 are hereby substantially simultaneously clamped, and thus fixed, between a respective clamping section of the clamping sleeve 63, on the one hand, and the inner wall of the nail 11 bounding the respective transverse bore 13, on the other hand.

The securing screw 73 preferably manufactured of plastic is made such that it is deformed or sheared off by this axial displacement of the clamping sleeve 63 and such that the fixing of the condyle screws 15 is thus not impaired by the securing screw 73. The securing screw 73 can, for example, consist of a biocompatible resorbable polylactate.

To ensure that all condyle screws 15 are securely fixed, the clamping sleeve 63 is preferably made of a material which can be deformed in the axial direction by the drawing forces which can be applied by means of the drawing screw 67.

So that the condyle screw 15 disposed furthest away from the drawing screw 67 comes into clamping engagement with the clamping sleeve 63 first after the start of the drawing movement, the passages 69 of the clamping sleeve 63 can be positioned such that they are aligned with the transverse bores 13 of the nail 11 in a pre-loaded state of the clamping sleeve 63 in which the clamping sleeve 63 is pressed toward the axial end abutment 79 of the nail 11 and is compressed at least slightly in the axial direction.

An advantage of the clamping sleeve 63 provided for a plurality of screws 15 consists in the fact that the clamping of the condyle screws 15 can be released again in a particularly simple manner in that the drawing screw 67 is unscrewed a little and the clamping sleeve 63 is thereby somewhat relieved, whereupon all clamping connections between the clamping sleeve 63 and the condyle screws 15 areh released simultaneously by a light tap on the head of the drawing screw 67.

### REFERENCE NUMERAL LIST

11 nail
13 transverse bore
15 screw
35 longitudinal bore of the nail
36 inner thread section of the nail
61 clamping member, grub screw
62 outer thread of the grub screw
63 clamping member, clamping sleeve
64 passage of the insert
65 insert
66 inner thread section of the insert
67 adjustment device, drawing screw
68 drawing section of the adjustment device
69 passage of the clamping sleeve
71 thread section of the clamping sleeve
73 securing member, securing screw
75 recess of the clamping sleeve
77 passage of the nail
79 end abutment of the nail

1. A bone fixing system comprising at least one nail (11), and at least one screw (15), which can be guided through a transverse bore (13) formed in the nail (11) and defining the orientation and the position of the screw (15) with respect to the longitudinal axis of the nail (11),
wherein at least one clamping member (61, 63) which can be introduced into a longitudinal bore (35) of the nail (11) and is axially adjustable in the longitudinal bore (35) relative to the nail (11), with the screw guided through the transverse bore (13) of the nail (11) being able to be clamped between the clamping member (61, 63) and the inner wall of the nail (11) bounding the transverse bore (13) by the displacement of the clamping member (61, 63).

2. A bone fixing system in accordance with claim 1, wherein the longitudinal bore (35) of the nail (11) is provided with an inner thread section (36) in which the clamping member (61) can be screwed.

3. A bone fixing system in accordance with claim 1, wherein the clamping member (61) is made in one piece and is provided in the form of a grub screw.

4. A bone fixing system in accordance with claim 1, wherein at least one sleeve-like or bushing-like insert (65) is inserted into the longitudinal bore (35) of the nail (11) and has at least one passage (64) aligned with the transverse bore (13) of the nail (11) and with which the clamping member (61) cooperates.

5. A bone fixing system in accordance with claim 4, wherein the inner side of the insert (65) is provided with an inner thread section (66) in which the clamping member (61) can be screwed.

6. A bone fixing system in accordance with claim 4, wherein the insert (65) is made of a first material, a cobalt chromium alloy, which has a higher toughness and/or hardness than a second material, titanium or a titanium alloy, of the nail (11).

7. A bone fixing system in accordance with claim 4, wherein the insert (65) is rotationally fixedly connected to the nail (11).

8. A bone fixing system in accordance with claim 4, wherein the insert (65) is pressed or screwed into the longitudinal bore (35) of the nail (11).

9. A bone fixing system in accordance with claim 1, wherein a plurality of transverse bores (13) are formed in the nail (11) and a clamping member (61) is provided for each screw (15) which can be guided through one of the transverse bores (13).

10. A bone fixing system in accordance with claim 1, having a plurality of nails (11), wherein a set of different axial spacings is provided between the nails (11) having transverse bores (13) and the axial length of the clamping members (61) is respectively smaller than the smallest axial spacing between two sequential transverse bores (13) occurring in the set.

11. A bone fixing system in accordance with claim 1, wherein a section of the clamping member (63) disposed on the side of the screw (15) remote from the displacement device (67) can be moved against the screw (15) by means of a displacement device (67) by pulling on the clamping member (63).

12. A bone fixing system in accordance with claim 11, wherein the clamping member (63) is freely movable at least in the axial direction in the longitudinal bore (35) of the nail (11) and has at least one passage (69) for the screw (15) which can be aligned with the transverse bore (13) of the nail (11), with the clamping member (63) being made in sleeve shape.

13. A bone fixing system in accordance with claim 11, wherein the displacement device (67) includes a drawing screw which cooperates with a thread section (71) of the clamping member (63) and is supported at the nail (11) for the drawing of the clamping member (63) in the axial direction.

14. A bone fixing system in accordance with claim 11, wherein the clamping member (63) has a plurality of passages (69) which are spaced apart from one another in the axial direction and can each be aligned with a transverse bore (13) of the nail (11).

15. A bone fixing system in accordance with claim 11, wherein the clamping member (63) can be deformed in the axial direction by means of the displacement device (67).

16. A bone fixing system in accordance with claim 15, having a plurality of screws (15), wherein the clamping member (63) can be deformed such that the plurality of screws (15) spaced apart from one another in the axial direction of the nail (11) can each be clamped between the clamping member (63) and the inner wall of the nail (11) bounding the respective transverse bore (13) by the displacement.

17. A bone fixing system in accordance with claim 11, wherein at least one securing member (73), a securing screw, is provided which can be moved from the outside through the side wall of the nail (11) into its longitudinal bore (35) and by which the clamping member (63) can be fixed in its starting position relative to the nail (11) prior to the actuation of the displacement device (67).
